(19) United States
(12) Patent Application Publication
(10) Pub. No.: US 2006/0122403 A1
(43) Pub. Date: Jun. 8, 2006

(54) Atorvastatin calcium form vi or hydrates thereof

(76) Inventors: Sanjay Suri, New Delhi (IN); Jujhar Singh, New Delhi (IN); Singh Manmohan Grewal, New Delhi (IN); Baldev Raj, New Dehli (IN)

Correspondence Address: Louis M Heidelberger;Reed Smith, 2500 One Liberty Place, 1650 Market Street, Philadelphia, PA, 19103, US

(21) Appl. No.: 10/520,020

(22) PCT Filed: Sep. 3, 2002
(86) PCT No.: PCT/IN02/00180

§ 371 Date: Oct. 3, 2005

(51) Int. Cl. C07D 207/34

(52) U.S. Cl. 548/537

(57) ABSTRACT

Atorvastatin calcium Form VI Or hydrates thereof, characterized by its X-ray powder diffraction and/or solid state NMR is described, as well as methods for the preparation of the same.

### FIELD OF THE INVENTION

The present invention relates to Atorvastatin Calcium Form VI or hydrates thereof and a process for preparing it. Particularly the invention relates to a novel crystalline form of Atorvastatin calcium.

### BACKGROUND OF THE INVENTION

Atorvastatin is a member of the class of drugs called statins. Statins drugs are currently the most therapeutically effective drugs available for reducing low density lipoprotein (LDL) particle concentration in the blood stream of patients at risk for cardiovascular disease. It also appears to reduce total glycerides and total cholesterol. A high level of LDL in the blood stream has been linked to the formation of coronary lesions, which obstruct the flow of blood and can rupture and promote thrombosis. Goodman and Gilman. The Pharmacological Basis of Therapeutics 879 (9^{th} ed. 1996). Reducing plasma LDL levels has been shown to reduce the risk of clinical events in patients with cardiovascular disease and patients who are free of cardiovascular disease but who have hypercholesterolemia. [Scandinavian Simvastatin Survival Study Group, 1994; Lipid Research Clinics Program, 1984a, 1984b].

The mechanism of action of statin drugs has been elucidated in some detail. They interfere with the synthesis of cholesterol and other sterols in the liver by competitively inhibiting the 3-hydroxy-3-methyl-glutaryl-coenzyme A reductase enzyme (“HMG-CoA reductase”). HMG-CoA reductase catalyzes the conversion HMG-CoA to mevalonate, which is the rate determining step in the biosynthesis of cholesterol, and so, its inhibition leads to a reduction in the concentration of cholesterol in the liver. Very low density lipoprotein (VLDL) is the biological vehicle for transporting cholesterol and triglycerides from the liver to peripheral cells. VLDL is catabolized in the peripheral cells which releases fatty acids which may be stored in adipocytes or oxidized by muscle. The VLDL is converted to intermediate density lipoprotein (IDL), which is either removed by an LDL receptor, or is converted to LDL. Decreased production of cholesterol leads to an increase in the number of LDL receptors and corresponding reduction in the production of LDL particles by metabolism of IDL.

Atorvastatin is the common chemical name of [R—(R*, R*)]-2-(4-fluorophenyl)-β,δ-dihydroxy-5-(1-methylethyl)-3-phenyl-4-[(phenylamino)carbonyl]-1H-pyrrole- 1-heptanoic acid. The free acid is prone to lactonization. The molecular structure of the lactone is represented by formula (I).

Atorvastatin is marketed as the hemi calcium salt-trihydrate under the name LIPITOR by Warner-Lambert Co. It is a synthetic HMG-CoA reductase inhibitor and is used for the treatment of hyperlipidemia and hypercholestorlemia. The empirical formula of Atorvastatin calcium is (C₃₃H₃₄FN₂O₅)₂Ca and its molecular weight is 1155.42.

Its structural formula is:

[image]

Atorvastatin calcium is a white to off white amorphous or crystalline powder that is insoluble in aqueous solutions of pH 4 and below. It is very slightly soluble in distilled water, pH 7.4 phosphate buffer, and acetonitrile, slightly soluble in ethanol, and freely soluble in methanol.

Atorvastatin lactone was first disclosed to the public and claimed in U.S. Pat. No. 4,681,893. The hemi calcium salt depicted in formula (II) (hereafter “atorvastatin calcium”) an enantiomer having R— form of the ring opened acid is disclosed in U.S. Pat. No. 5,273,995. This patent teaches that the calcium salt is obtained by crystallization from a brine solution resulting from the transposition of the sodium salt with CaCl₂ and further purified by recrystallization from a 5:3 mixture of ethyl acetate and hexane. Both of these U.S. patents are hereby incorporated by reference.

U.S. Pat. Nos. 5,003,080; 5,097,045; 5,103,024; 5,124,482; 5,149,837; 5,155,251; 5,216,174; 5,248,793; 5,280,132; 5,342,952; 5,007,080; 6,274,740; which are herein incorporated by reference describe various processes and key intermediates for preparing atorvastatin calcium. All these processes give mixture of crystalline and amorphous forms.

Atorvastatin is prepared as its calcium salt. The calcium salt is desirable since it enables atorvastatin to be conveniently formulated in for administration purposes. Additionally, there is a need to produce atorvastatin in a pure and crystalline form to enable to meet exacting pharmaceutical requirements and specifications.

Furthermore, the process by which atorvastatin is produced needs to be one which is amenable to large scale production. Additionally, it is desirable that the product should be in form that is readily isolated. Finally, it is economically desirable that the product should have long shelf life without the need for specialized storage conditions.

The processes in the above United States patents disclose amorphous atorvastatin, which has unsuitable filtration and drying characteristics for large-scale production and must be protected from heat, light, oxygen and moisture.

U.S. Pat. No. 5,969,156 discloses three polymorphs of atorvastatin designated Forms I, II, and IV by the inventors of those forms. Though the inventors claim certain processing and therapeutic advantages of their forms over the amorphous atorvastatin calcium, advantages may yet be realized by other heretofore undiscovered forms of atorvastatin calcium.

PCT application WO 97/03960 and PCT application WO 00/71116 describes method for the production of amorphous atorvastatin calcium.

PCT application WO97/03958 and U.S. Pat. No. 6,121,461 disclose the method for the preparation of Form III crystalline atorvastatin calcium while PCT application WO97/03959 teaches a method for the preparation of Form I, II, and IV of crystalline atorvastatin calcium.

PCT application WO01/36384 discloses Form V of atorvastatin calcium. All these patents claim advantages over the existing patents in one way or the other.

The present invitation includes a new crystal form of atorvastatin calcium in both hydrate and anhydrous states. Polymorphism is the property of some molecules and molecular complexes to assume more than one crystalline or amorphous form in the solid state. A single molecule, like the atorvastatin in formula (I) or the salt complex of formula (II), may give rise to a variety of solids having distinct physical properties like solubility, stability, purity, X-ray diffraction pattern and solid state ¹³C NMR spectrum. The differences in the physical properties of polymorphs result from the orientation and intermolecular interactions of adjacent molecules (complexes) in the bulk solid. Accordingly, polymorphs are distinct solids sharing the same molecular formula, which may be thought of as analogous to a unit cell in metallurgy, yet having distinct advantageous and/or disadvantageous physical properties compared to other forms in the polymorph family. One of the most important physical properties of pharmaceutical polymorphs is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. On the other hand, where the effectiveness of a drug correlates with peak bloodstream levels of the drug, a property shared by statin drugs, and provided the drug is rapidly absorbed by the GI system, then a more rapidly dissolving form is likely to exhibit increased effectiveness over a comparable amount of a more slowly dissolving form.

### BRIEF DESCRIPTION OF THE DRAWING

FIG. 1 denotes structural formula of a new crystalline polymorphic Form VI of atorvastatin calcium of the present invention.

FIG. 2 is an X-ray powder diffractogram of atorvastatin calcium Form VI.

FIG. 3 is a solid state C¹³ NMR spectrum of atorvastatin calcium Form VI.

### SUMMARY OF THE INVENTION

The present invention provides new crystalline polymorphic form of atorvastatin calcium designated as Form VI in both anhydrous and hydrate states, which possess the advantage of higher purity, stability and solubility.

The present invention further provides a simple cost effective process for preparing new Form VI of atorvastatin calcium that has merits of easy and rapid isolation & crystallization without comprising the purity, yield and stability. The number of steps involved is very few.

The invention also results in high yield, and very low volume of residual solvents.

Accordingly the present invention is directed to crystalline polymorphic Form VI of atorvastatin calcium both in anhydrous and hydrate states thereof.

The new polymorphic crystalline Form VI of atorvastatin calcium is characterized by the following X-ray powder diffraction pattern expressed in terms of the 2 theta, d-spacings, and relative intensities with a relative intensity of >15% measured on a Shimadzu XRD-6000 with copper K radiation of lamda 1.5406° A:

| 2θ | D | Relative intensity (>15%) |
|---|---|---|
| 3.7365 | 23.4584 | 23.0 |
| 7.7200 | 11.4425 | 36.0 |
| 8.6985 | 10.1574 | 74.0 |
| 10.2185 | 8.6497 | 57.0 |
| 12.5933 | 7.0234 | 19.0 |
| 17.9103 | 4.9485 | 47.0 |
| 18.3600 | 4.8283 | 20.0 |
| 19.4031 | 4.5710 | 100.0 |
| 20.2800 | 4.3753 | 29.0 |
| 20.8200 | 4.2630 | 48.0 |
| 22.5122 | 3.9463 | 24.0 |
| 25.5848 | 3.4923 | 25.0 |

Further, the crystalline Form VI atorvastatin calcium or hydrates thereof of claim 1 having X-ray powder diffraction peaks at 3.7, 8.6, 10.2 and 20.9 degrees at 2 theta and a broad peak at 19.5 degree 2 theta.

Further the present invention is directed to crystalline Form VI atorvastatin and hydrates thereof characterized by the following solid state C¹³ nuclear magnetic resonance spectrum (NMR) wherein chemical shift is expressed in parts per million (PPM) measured on Varian spectrophotometer:

δ(ppm)

21.898
24.294
27.767
29.368
33.939
38.275
42.836
45.980
68.932
71.266
73.617
119.357
122.987
131.214
137.515
162.696
169.066
179.540
186.890
190.640

The Crystalline Form VI atorvastatin calcium or hydrates thereof of claim 1 having solid state C¹³ NMR signals at about 162.689 ppm, 169.066 ppm, 179.54 ppm, 186.89 ppm, and 190.64 ppm.

In a preferred embodiment of the present invention crystalline Form VI of atorvastatin calcium contains up to 8 moles of water per mole of atorvastatin calcium.

In a still preferred embodiment of the present invention crystalline Form VI of atorvastatin calcium is trihydrate.

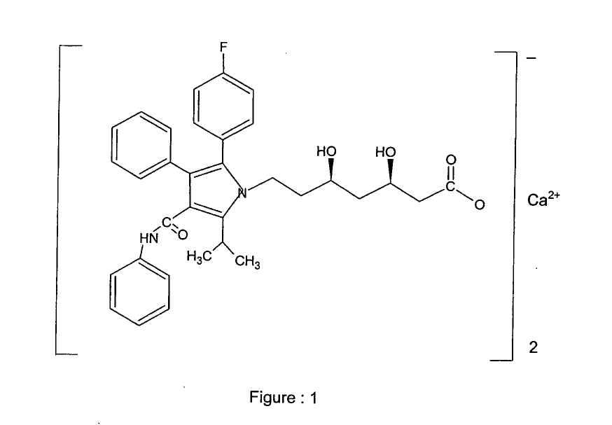

The present invention further provides a process for the preparation of new crystalline polymorphic Form VI of atorvastatin calcium both hydrate and anhydrous states, [R—(R*, R*)]-2-(4-flurophenyl)-beta,delta-dihydroxy-5-(1-methylethyl)-3-phenyl-4-[(phenyl amino)carbonyl]-1H-pyrrole-1-heptanoic acid hemicalcium salt (2:1) having formula as shown in FIG. 1 of the drawing accompanying this specification which comprises:
a) dissolving calcium salt of any form of atorvastatin in an organic solvent such as aliphatic ketone to get clear solution of atorvastatin salt,
b) optionally removing impurities,
c) adding demineralised water,
d) isolating crystallized polymorphic Form VI of atorvastatin calcium and drying, if desired, to get required water of crystallization.

Further the present invention also provide a process for the preparation of new polymorphic crystalline Form VI of atorvastatin calcium, [R—(R*, R*)]-2-(4-fluorophenyl)-beta,delta-dihydroxy-5-(1-methylethyl)-phenyl-4-[(phenylamino)carbonyl]-1H-pyrrole-1-heptanoic acid calcium salt (2:1) having formula of FIG. 1 which comprises:
a) dissolving lactone form of atorvastatin in an organic solvent preferably aliphatic ketone to get a clear solution,
b) adding an aqueous solution of alkaline solution of earth metal hydroxide and demineralised water under stirring,
c) isolating crystallized polymorphic Form VI of atorvastatin calcium and drying, if desired, to get required water of crystallization.

In an embodiment of the present invention, the atorvastatin calcium used may be amorphous or crystalline Form I, II, III, IV, & V of atorvastatin calcium or mixture thereof.

In a further embodiment, atorvastatin calcium used may be in anhydrous or hydrate state containing up to 9 water molecules.

In a still further embodiment an organic solvent used may be selected from aliphatic ketones having 1 to 3 carbon atoms. The aliphatic ketones used may be acetone, methyl ethyl ketone, diethyl ketone, methyl propyl ketone, preferably acetone.

In yet another embodiment, the organic solvent used may be 100 times preferably 15 times more preferably 10 times of the starting compound.

According to the further aspect of the invention, the dissolution may be carried out by heating the suspension of atorvastatin calcium in an organic solvent to the reflux temperature of the solvent used preferably above 40 and below 80° C. more preferably 40 to 50° C.

In a further embodiment the impurities may be removed by filtration.

In yet another embodiment the DM water used may be 100 times preferably 10 times more preferably 5 times of the starting compound.

In another embodiment DM water may be added drop wise maintaining the temperature.

Further the alkaline earth metal hydroxide used may be calcium hydroxide. The aqueous solution of earth metal hydroxide may preferably be added at elevated temperature preferably above 40° C. and below 80° C. more preferably at 40 to 50° C.

The alkaline earth metal hydroxide may be added 50 times preferably 10 times of the starting compound more preferably in 1:1 ratio.

In still another embodiment the solution may be cooled slowly to a temperature in the range of −20° C. to 20° (room temperature) preferably in the range of 15 to 20° C. to effect crystallization. The cooling may be effected @ of 2 to 3° C.

The isolation may be effected by any conventional methods such as filtration, vacuum filtration, decantation, centrifugation.

The drying may be effected by known means like vacuum tray drier, Rotacon vacuum drier, and at a temperature above 50 and below 80° C., preferably at 55° C. for 12 to 30 hours to regulate the water of molecules. One skilled in the art will appreciate that by adjusting the temperature and time for these steps one can optimize the yield of the desired product.

The new crystalline Form VI atorvastatin calcium has potential use for the treatment of hyperlipidemia, hypercholesteromelia, hypocholesterolemia, alzheimer's disease atherosclerosis, xanthoma, and in synergism with other drugs for treatment of phytosterolemia lipase deficiency and the like.

### DETAILED DESCRIPTION OF THE INVENTION

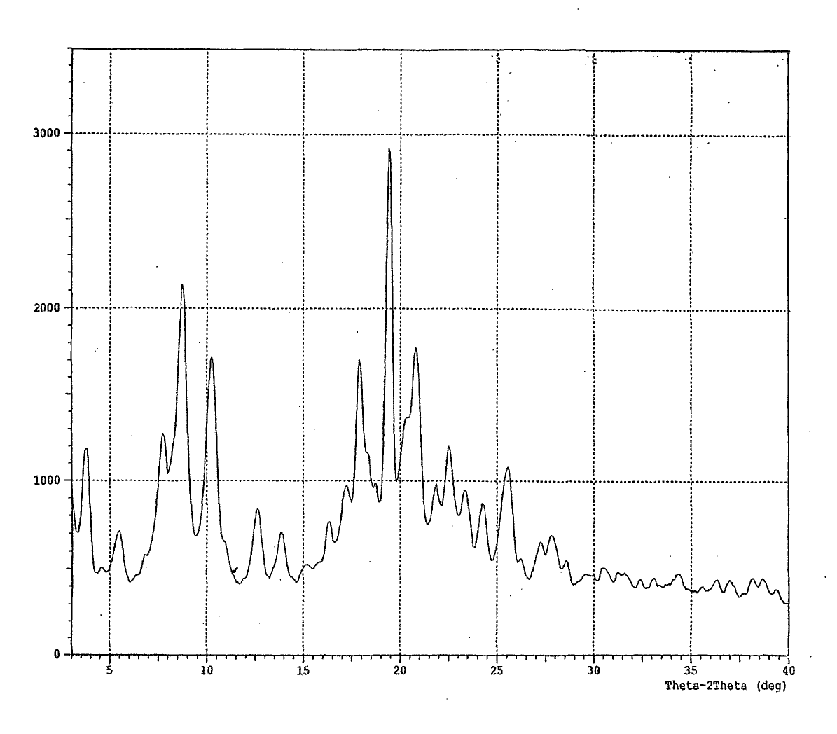

The X-ray powder diffractogram of new polymorphic crystalline Form VI (FIG. 2) has medium peaks at 3.7±0.2, 8.6±0.2, 10.2±0.2 and 20.9±0.2 degree 2-θ and one large peak at 19.5±0.2 degree 2-θ.

This X-ray pattern is well distinguished from that of known crystalline Forms I, II, III, IV, V and also from the X-ray pattern of amorphous form, which is characterised by two broad humps in the ranges 8-14 degree 2-θ and 15-26 degree 2-θ.

The X-ray powder diffractogram of FIG. 2 was obtained by known methods using a Shimadzu XRD-6000, copper radiation of λ=1.5406° A was used. Measurement range 3-40 degree 2-θ. Table 1 list the 2-θ, d-spacings and relative intensities with a relative intensity of >15%.

TABLE 1

| 2-θ | d | Relative Intensity (>15%) |
|---|---|---|
| 3.7635 | 23.4584 | 23.0 |
| 7.7200 | 11.4425 | 36.0 |
| 8.6985 | 10.1574 | 74.0 |
| 10.2185 | 8.6497 | 57.0 |
| 12.5933 | 7.0234 | 19.0 |
| 17.9103 | 4.9485 | 47.0 |
| 18.3600 | 4.8283 | 20.0 |
| 19.4031 | 4.5710 | 100.0 |
| 20.2800 | 4.3753 | 29.0 |
| 20.8200 | 4.2630 | 48.0 |
| 22.5122 | 3.9463 | 24.0 |
| 25.5848 | 3.4923 | 25.0 |

The solid state C¹³ NMR spectrum of new polymorphic form is characterised by the following chemical shifts.

```
δ (ppm)
  21.898
  24.294
  27.767
  29.368
  33.939
  38.275
  42.836 (strong)
  45.980
  68.932
  71.266
  73.617
119.357
122.987
131.214 (strong)
137.515
162.696
169.066169.066
179.540
186.890
190.640
```

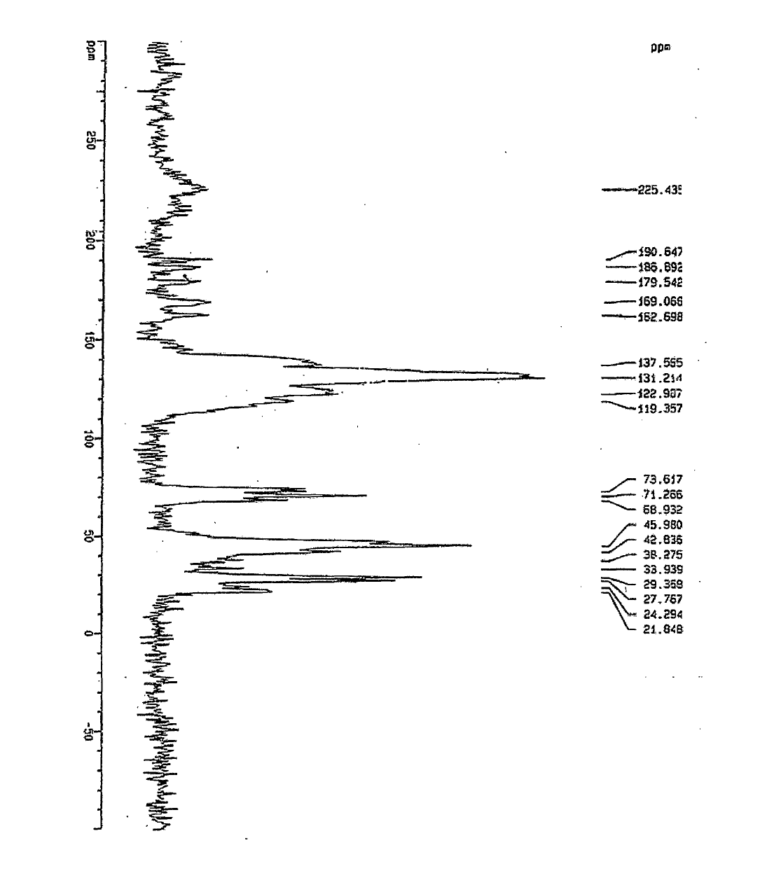

The solid state C¹³ NMR spectrum is well distinguished from those of known Forms I, II, III, IV, V and also amorphous form which display a different pattern with shifts significantly different from that of new polymorphic Form VI claimed at 162.698 ppm, 169.066 ppm, 179.54, 186.89 ppm and 190.64 ppm which corresponds to C₁₂ or C₂₅ carbons of compound of formula of FIG. 1. The spectrum of FIG. 3 was obtained on Varian spectrometer operating at 300 MH_{z}. The instrument was equipped with a 13C cp mass probe head and sample was spun at 7.0 KH_{z} spin rate. The magic angle and proton decoupling efficiency were optimised before acquisition.

XRD and NMR were performed on ungrounded samples.

The new polymorphic form exits in anhydrous as well as hydrous form. It contains up to 9 water molecules. However, trihydrate form is preferable.

The invention is further illustrated by the following examples, which do not limit the effective scope of the claims.

### EXAMPLE 1

Atorvastatin Calcium (100.0 g) was added to acetone (1.0 Ltr.) at room temperature. The mixture was heated at 50° C. for 30 minutes to get clear solution. DM-Water (500 ml) was added drop wise to this solution at 50° C. The solution was slowly cooled to room temperature at rate of 2° C./minute during which new polymorphic form of Atorvastatin Calcium crystallises out. The product is filtered by vacuum filtration and then dried in vacuum tray drier at 50-55° C. for 24 hours.

| | |
|---|---|
| Yield | 90.0 gm (90.0%) |
| Relative purity (HPLC) | 99.63% |
| Residual solvent | |
| Acetone | NMT 0.2% |

### EXAMPLE 2

Atorvastatin Calcium (100.0 g) was added into acetone (100.0 ml) at room temperature. The mixture was heated at 50° C. for 30 minutes to get clear solution. DM-Water (100 ml) was added drop wise to this solution at 50° C. The solution was slowly cooled to room temperature at rate of 2° C./minute during which new polymorphic form of Atorvastatin Calcium crystallises out. The product is filtered by vacuum filtration and then dried in vacuum tray drier at 55-60° C. for 28 hours.

| | |
|---|---|
| Yield | 92.0 gm (92.0%) |
| Relative purity (HPLC) | 99.68% |
| Residual solvent | |
| Acetone | NMT 0.2% |

### EXAMPLE 3

Atorvastatin Calcium (10.0 g) was added into acetone (1.0 Ltr.) at room temperature. The mixture was heated at 45° C. for 20 minutes to get clear solution. DM-Water (1.0 Ltr.) was added drop wise to this solution at 45° C. The solution was slowly cooled to room temperature at rate of 2° C./minute during which new polymorphic form of Atorvastatin Calcium crystallises out. The product is filtered by vacuum filtration and then dried in vacuum tray drier at 55-60° C. for 24 hours.

| | |
|---|---|
| Yield | 90.0 gm (90.0%) |
| Relative purity (HPLC) | 99.61% |
| Residual solvent | |
| Acetone | NMT 0.2% |

### EXAMPLE 4

Lactone form of Atorvastatin Calcium (100.0 g) was added into acetone (1.0 Ltr.) at room temperature. To this was added calcium hydroxide (10.0 g) suspended in DM-Water (100 ml) in one lot. The reaction mass was stirred at 45-46° C. till disappearance of lactone form of Atorvastatin Calcium (TLC, 2.0 hrs.). DM-Water (400 ml) was added drop wise at 45° C. The solution was slowly cooled to room temperature at rate of 2° C./minute during which new polymorphic form of Atorvastatin Calcium crystallises out. The product is filtered by vacuum filtration and then dried in vacuum tray drier at 50-55° C. for 20 hours.

| | |
|---|---|
| Yield | 100.0 gm (90.0%) |
| Relative purity (HPLC) | 99.31% |
| Residual solvent | |
| Acetone | NMT 0.2% |

### EXAMPLE 5

Lactone form of Atorvastatin Calcium (10.0 g) was added into acetone (10.0 ml.) at room temperature. To this was added calcium hydroxide (1.0 g) suspended in DM-Water (5 ml) in one lot. The reaction mass was stirred at 50° C. till disappearance of lactone form of Atorvastatin Calcium (TLC, 2.0 hrs.). DM-Water (5 ml) was added drop wise at 50° C. The solution was slowly cooled to room temperature at rate of 2° C./minute during which new polymorphic form of Atorvastatin Calcium crystallises out. The product is filtered by vacuum filtration and then dried in vacuum tray drier at 55-60° C. for 24 hours.

| | |
|---|---|
| Yield | 10.0 gm (90.0%) |
| Relative purity (HPLC) | 99.20% |
| Residual solvent | |
| Acetone | NMT 0.2% |

### EXAMPLE 6

Lactone form of Atorvastatin Calcium (10.0 g) was added into acetone (1.0 Ltr.) at room temperature. To this was added calcium hydroxide (1.0 g) suspended in DM-Water (100 ml) in one lot. The reaction mass was stirred at 45-46° C. till disappearance of lactone form of Atorvastatin Calcium (TLC, 2.0 hrs.). DM-Water (900 ml) was added drop wise at 45-46° C. The solution was slowly cooled to room temperature at rate of 2° C./minute during which new polymorphic form of Atorvastatin Calcium crystallises out. The product is filtered by vacuum filtration and then dried in vacuum tray drier at 55-60° C. for 24 hours.

| | |
|---|---|
| Yield | 10.2 gm (92.0%) |
| Relative purity (HPLC) | 99.22% |
| Residual solvent | |
| Acetone | NMT 0.2% |

While the present invention has been described in terms of its specific embodiments, certain modifications and equivalents will be apparent to those skilled in the art and are intended to be included within the scope of the present invention.

We claim:

1. A crystalline Form VI atorvastatin calcium or hydrates thereof having characterized by the X-ray powder diffraction pattern following 2θ values measured using a Shimadzu XRD-6000 with copper K radiation of λ1.5406° A and with a relative intensity of >15%
3.7365, 7.7200, 8.6985, 10.2185, 12.5933, 17.9103, 18.3600, 19.4031, 20.2800, 20.8200, 22.5122, and 25.5848

2. A crystalline Form VI atorvastatin calcium or hydrates thereof of claim 1 having X-ray powder diffraction peaks at about 3.7, 18.0, and 20.9 degrees at 2-θ and large peaks at 8.6, 10.2, and 19.5 degree 2-θ.

3. A crystalline Form VI atorvastatin calcium or hydrates thereof of claim 1 having characterized by the following solid state C¹³ nuclear magnetic resonance spectrum (NMR) wherein chemical shift is expressed in parts per million (PPM):
δ (ppm)
21.898
24.294
27.767
29.368
33.939
38.275
42.836
45.980
68.932
71.266
73.617
119.357
122.987
131.214
137.515
162.696
169.066
179.540
186.890
190.640

4. A crystalline Form VI atorvastatin calcium or hydrates thereof of claim 1 having solid state C¹³ NMR signals at about 162.689 ppm, 169.066 ppm, 179.54 ppm, 186.89 ppm, and 190.64 ppm.

5. A crystalline Form VI atorvastatin calcium of claim 1 contains up to 8 moles of water per mole of atorvastafin calcium.

6. A crystalline Form VI atorvastatin calcium of claim 1 contains up to 3 moles of water per mole of atorvastatin calcium.

7. A crystalline Form VI atorvastatin calcium of claim 1 has melting point in the range of 177 to 182° C.

8. A process for the preparation of a crystalline Form VI atorvastatin calcium of claim 1 both hydrate and anhydrous states, [R—(R*, R*)]-2-(4-flurophenyl)-beta,delta-dihydroxy-5-(1-methylethyl)-3-phenyl-4-[(phenyl amino)carbonyl]-1H-pyrrole-1-heptanoic acid hemicalcium salt (2:1) having formula as shown in FIG. 1 of the drawing accompanying this specification which comprises:
a) dissolving calcium salt of any form of atorvastatin in an organic solvent such as aliphatic ketone preferably at a temperature in the range of ambient to reflux temperature to get clear solution of atorvastatin salt,
b) optionally removing impurities,
a) adding demineralised water maintaining the same temperature,
d) isolating crystallized polymorphic Form VI of atorvastatin calcium and drying, if desired, to get required water of crystallization.

9. A process for the preparation of new polymorphic crystalline Form VI of atorvastatin calcium,[R—(R*, R*)]-2-(4-fluorophenyl)-beta,delta-dihydroxy-5-(1-methylethyl)-phenyl-4-[(phenylamino)carbonyl]-1H-pyrrole-1-heptanoic acid calcium salt (2:1) having formula of FIG. 1 which comprises:
a) dissolving lactone form of atorvastatin in an organic solvent preferably aliphatic ketone at a temperature in the range of ambient to reflux temperature to get a clear solution,
b) adding ah aqueous solution of alkaline solution of earth metal hydroxide and demineralised water under stirring maintaining the same temperature,
c) isolating crystallized polymorphic Form VI of atorvastatin calcium and drying, if desired, to get required water of crystallization.

10. A process of claims 8 & 9 wherein the atorvastatin calcium used is amorphous or crystalline Form I, II, III, IV, & V of atorvastatin calcium or mixture thereof.

11. A process of claims 8 & 9 wherein the atorvastatin calcium used is in anhydrous or hydrate state containing up to 9 water molecules.

12. A process of claims 8 & 9 wherein an organic solvent used is selected from aliphatic ketones having 1 to 3 carbon atoms.

13. A process of claims 8, 9 and 12 wherein the aliphatic ketones used are acetone, methyl ethyl ketone, diethyl ketone, methyl propyl ketone, preferably acetone.

14. A process of claims 8 & 9 wherein the organic solvent used is 100 times preferably 15 times more preferably 10 times of the starting compound.

15. A process of claims 8 & 9 wherein the dissolution is carried out by heating the suspension of atorvastatin calcium in an organic solvent to above 40 and below 80° C. more preferably 40 to 50° C.

16. A process of claims 8 & 9 wherein the impurities are removed by filtration.

17. A process of claims 8 & 9 wherein the demineralised (DM) water used is 100 times preferably 10 times more preferably 5 times of the starting compound.

18. A process of claim 9 wherein the alkaline earth metal hydroxide used is calcium hydroxide.

19. A process of claim 9 wherein the alkaline earth metal hydroxide added is 50 times preferably 10 times of the starting compound more preferably in 1:1 ratio.

20. A process of claims 8 & 9 wherein the cooling is effected slowly to a temperature in the range of −20° C. to 20° (room temperature) preferably in the range of 15 to 20° C. to effect crystallization. The cooling may be effected @ of 2 to 3° C.

21. A process of claims 8 & 9 wherein the isolation is carried out conventional methods such as filtration, vacuum filtration, decantation, centrifugation.

22. A process of claims 8 & 9 wherein the drying is effected by known means like vacuum tray drier, rotacon vacuum drier, and at a temperature above 50 and below 80° C., preferably at 55° C. for 12 to 30 hours.
